SEQUENCE LISTING (29 sequences)

SEQ ID NO: 1 - DNA, 2976 bp, Homo sapiens
  CDS (1347)..(2648): GPR22
gtcattctgcaaaagctgcccgaacaggctttcctctgaaaagcagtgccgttcctttttagaaagactgaatctaaatgttgtctctggagacaagaagccttcagtatgttaaattactttcattatgtattttcagatgcttattgattccacagtaggaagagtgagagactgcagcagcctctaagcagcactacatgttccatacattagcagtactgctgaaacaatggcacactacagacacatattttcattaaggtcattttcgaagagatgtttatgaccctcttcccccagtcctctactaacaagtgaacaaaatgaatcaatccaaactggaaatgcttcaactacatcaagaatttatcaaatctttagcagagggccaatagcagctaattcagtatttacaactgacaatatgaagaatgcaattgactgagcatctccctagctgtctgaactacgaactgcaagatgttcttgtaacacgactttaagacattaaggagttaaaaccagggaataggtctacattactgatggaatataaaaaatcaactgtatcctaagaagatgctacataaaataaccacaaaaagaaaaacaatataactgtaaaagcctgaaaagaatttttaaaaggggaagtttatactttcatataccagaattgtggaagttactgattctggaagacataatgaaacatgaatttccaaaaagaaaagaaaatactttatcagcacacaaaaggaagatttaggaagtgttttctgcactaaatattcagatattcatatcaattggtatgactaatggatttttctatctgacttttatgaccaattatgtatcctcttctaatgaaaacaaacaaaattaaacagcagatggtttttatcaaaaggacatggcctggatttataatataaagcaagttatgtgatcaagaaatcatttcaaaatagtgagcactgctattaaaaacagatttacaatggtaacaaaaggatgtctaaatatattttagaagctacaaacgttatgtttccttttttgttttcacatattctggaaaataaagaaatattatcatgtactccatcaaagggaaacataattcctatcatctgaggaaattcctcttggccgtgactttttaaagcaaaacaaatacaaatattatgtactgttctttagaaatccatcagccaactaaatctcataatgcatgcagttgaagtattggagagaaaacgaaagaattcctacaagacatgaaataaaacacagctacttcactgttgtcagggaaaaaaaccaactgctccaaaagaatgtgtttttctcccattctggaaatcaacatgcagtctgaatctaacattacagtgcgagatgacattgatgacatcaacaccaatatgtaccaaccactatcatatccgttaagctttcaagtgtctctcaccggatttcttatgttagaaattgtgttgggacttggcagcaacctcactgtattggtactttactgcatgaaatccaacttaatcaactctgtcagtaacattattacaatgaatcttcatgtacttgatgtaataatttgtgtgggatgtattcctctaactatagttatccttctgctttcactggagagtaacactgctctcatttgctgtttccatgaggcttgtgtatcttttgcaagtgtctcaacagcaatcaacgtttttgctatcactttggacagatatgacatctctgtaaaacctgcaaaccgaattctgacaatgggcagagctgtaatgttaatgatatccatttggattttttcttttttctctttcctgattccttttattgaggtaaattttttcagtcttcaaagtggaaatacctgggaaaacaagacacttttatgtgtcagtacaaatgaatactacactgaactgggaatgtattatcacctgttagtacagatcccaatattctttttcactgttgtagtaatgttaatcacatacaccaaaatacttcaggctcttaatattcgaataggcacaagattttcaacagggcagaagaagaaagcaagaaagaaaaagacaatttctctaaccacacaacatgaggctacagacatgtcacaaagcagtggtgggagaaatgtagtctttggtgtaagaacttcagtttctgtaataattgccctccggcgagctgtgaaacgacaccgtgaacgacgagaaagacaaaagagagtcttcaggatgtctttattgattatttctacatttcttctctgctggacaccaatttctgttttaaataccaccattttatgtttaggcccaagtgaccttttagtaaaattaagattgtgttttttagtcatggcttatggaacaactatatttcaccctctattatatgcattcactagacaaaaatttcaaaaggtcttgaaaagtaaaatgaaaaagcgagttgtttctatagtagaagctgatcccctgcctaataatgctgtaatacacaactcttggatagatcctaaaagaaacaaaaaaattacctttgaagatagtgaaataagagaaaaatgtttagtgcctcaggttgtcacagactagagaaaagtctcagtttcaccaaatccacattcaaatgagttttaaatttaaattgtaaaaactgatattactgccaaatataagaaaaatattttaagtattggttatgttgtaaattttcaatgtgaatgtcaattagataggtcatatatattcaatttcttcattacttaatgtatttgttgcatggcagtttgttaaagtactatcatgtgtatattttgtcaatattatgtccaacagaaaatattcatgtaagtcatattttttaaggaataaatacatagccttaaaacagtgtataactttaaaatgtaaaaaaaaaaaa

SEQ ID NO: 2 - protein, 433 aa, Homo sapiens
MCFSPILEINMQSESNITVRDDIDDINTNMYQPLSYPLSFQVSLTGFLMLEIVLGLGSNLTVLVLYCMKSNLINSVSNIITMNLHVLDVIICVGCIPLTIVILLLSLESNTALICCFHEACVSFASVSTAINVFAITLDRYDISVKPANRILTMGRAVMLMISIWIFSFFSFLIPFIEVNFFSLQSGNTWENKTLLCVSTNEYYTELGMYYHLLVQIPIFFFTVVVMLITYTKILQALNIRIGTRFSTGQKKKARKKKTISLTTQHEATDMSQSSGGRNVVFGVRTSVSVIIALRRAVKRHRERRERQKRVFRMSLLIISTFLLCWTPISVLNTTILCLGPSDLLVKLRLCFLVMAYGTTIFHPLLYAFTRQKFQKVLKSKMKKRVVSIVEADPLPNNAVIHNSWIDPKRNKKITFEDSEIREKCLVPQVVTD

SEQ ID NO: 3 - DNA, 3669 bp, Homo sapiens
  CDS (18)..(917): PEX19
aagtcggaggtagcaagatggccgccgctgaggaaggctgtagtgtcggggccgaagcggacagggaattggaggagcttctggaaagtgctcttgatgatttcgataaagccaaaccctccccagcacccccttctaccaccacggcccctgatgcttcggggccccagaagagatcgccaggagacactgccaaagatgccctcttcgcttcccaagagaagtttttccaggaactattcgacagtgaactggcttcccaagccactgcggagttcgagaaggcaatgaaggagttggctgaggaagaaccccacctggtggagcagttccaaaagctctcagaggctgcagggagagtgggcagtgatatgacctcccaacaagaattcacttcttgcctaaaggaaacactaagtggattagccaaaaatgccactgaccttcagaactccagcatgtcggaagaagagctgaccaaggccatggaggggctaggcatggacgaaggggatggggaagggaacatcctccccatcatgcagagtattatgcagaacctactctccaaggatgtgctgtacccatcactgaaggagatcacagaaaagtatccagaatggttgcagagtcatcgggaatctctacctccagagcagtttgaaaaatatcaggagcagcacagcgtcatgtgcaaaatatgtgagcagtttgaggcagagacccccacagacagtgaaaccactcaaaaggctcgttttgagatggtgctggatcttatgcagcagctacaagatttaggccatcctccaaaagagctggctggagagatgcctcctggcctcaactttgacctggatgccctcaatctttcgggcccaccaggtgccagtggtgaacagtgtctgatcatgtgaaacacaacacgttttcctctctgagtcccagctatggggaacatctggagtcagcagaaccattgggacctgaggcaggagtgtcacctgcgggagaagtctgcccgctgccctctgtcatcccattcaagattgtgccataccagctgaggtttttcctctgtctctctaggaatagggtctgtttcacaggccatttctgtgaaccctactccattgtggtttctgccactatcaaagttccagctacctgcaaggtgaaggaaggcatcccttttggggcatgcactttctttcctttctcaaaataatgttatatgtggccacactgatgttcacctttacgtccagggtctttgtgccttgtctctactccctctcttggatctggggaggaggggcagagacctgggactctgtatttctatagttctcctggcagagcctttgagaatggggagaaacagcctgggctggggctacaggtctgtcactatgctctcttgccttcagacagaccattctgaattctctaaagggaaagggcttttgcatctaatcacaatagagttgaaagagaggccttaggattctcctctctctaggtgctgagccctcacctccctgttccaggctgagaactcaaatggttaccctgcttcttcctacaatgctgtgtgatatgggtgaacccagcccctgaccttcctctatcccctgcccatcctcccttttacctcctctcttttttaaacacctgtttatcccaacctttttgagctcaagctgtgataaagaagggcccatcctatttcccctcatctagtccatttacgattctcactgactccccgtcttcctggcagacacaaataaacccagtgtcaggtctaggaaattaatggctattcttccccagatacattctggcttatttgagatacatgattctcttagaatcctgtcccttggttcaggaaagtagcttggaaaaggagtaggggtatagcttgggtcccttttcctgcaaggccccatggggcagaatataataaatattctgagtgaggagtgtggtctttttctgatcttcctcagcttccgtaagttgcagagtgaggtatattaggagactagttctacacaatattgtaatgctgggttccatcaacacccaccttccacaactcagtctgcacctcagttggcaaaggagactggatggccatctttcctcatgttcccttgagtatttcaatgtagaaagcccttcaagtggtattatattttaaccttttacattattgttattaatgttagtaatatattgttatgttttctaaattatttttctttaagctgacgtggctttttttctgtggctcccagtgggtctacggaccttggctgacatatgttggtaggtactctggtcagctcagctggctgtcctggttcactcagaagataagtctctccaaagcaaattcacatgcattatgagtcgctttgagcttctgacatgtcacttgccccgaggttaaaacttttcaccccttgaagaccttacatgttttatggtattggtgaggaaggaaatgttctcaaggtctcaggctatttgggaaattccaactcctataccttaccagagcatggaagagcccagatctgaatgtaaaacgtctctgttctgccagagatggaaaaaatacaggtatacttgtgatatagtcatggggcttcagtgtcactattttctccttaaagctccagccaaaaactggacaaggatagagaggaggagggaagaacaaaagagcccttctctatgaaccttgtgccttctgtcctaccagttttcttttacagattctcacttctgctagcctagccagggcttactccaggaatctaaatagatgccctagtccactttatctttgttcccaaggcactcatttttattttgattttgattgaatgtgagcaggttgacctcaggtcacactttgttccaaaaacttttggaattattccaggacttgtggtggagttatggtactctagggcagtctttctcaaactatgtatggtaaaggaccaggttttttgttttccagtccttcacttatcaatatgcattcctattgccgatgacaggtatggagttcacactgtgtgctgccgacccggcaagtttgacagcacccaaactggccagactgttctgtaggttaagtccattgatcatgtacttggatatcacagcaacattgaaatgctaaaaagtttttaaacactctcaatttctaattcaccatgtcacagactggtgaaaaaaaaaaaaaggtgttcactgaccagcacaagtctgcagatcatctttgagtagcactgttttggggccctcggtctctctgaagaccctagcagaactgatacctacctgtatctcttgttctctcctatttgagtttcacttccagagaacttgttcttcagcaagaatgtgtcactagtaaggacatctctagcatttctctagccttccttttctgctgctcaaaaataatcgttacaaagcttaggtttaagctgtatatgaaatatttatgcgactctcaaactttaaaggagttgctcctttgttccaaaattaaatgtgttagataaatttgtgattgtatgggtggcttcatgaattaagaattgaattaatacagactttttgataataaaaaaaaaaaaa

SEQ ID NO: 4 - protein, 299 aa, Homo sapiens
MAAAEEGCSVGAEADRELEELLESALDDFDKAKPSPAPPSTTTAPDASGPQKRSPGDTAKDALFASQEKFFQELFDSELASQATAEFEKAMKELAEEEPHLVEQFQKLSEAAGRVGSDMTSQQEFTSCLKETLSGLAKNATDLQNSSMSEEELTKAMEGLGMDEGDGEGNILPIMQSIMQNLLSKDVLYPSLKEITEKYPEWLQSHRESLPPEQFEKYQEQHSVMCKICEQFEAETPTDSETTQKARFEMVLDLMQQLQDLGHPPKELAGEMPPGLNFDLDALNLSGPPGASGEQCLIM

SEQ ID NO: 5 - DNA, 5231 bp, Homo sapiens
  CDS (331)..(2208): GRHL2
agaaagttacctgtggccgcccaagtccgccactttctgctctgtgtctgcccattgccacgatccaggaggactccgcgccgcccggccgcctccgagctcgggccccatgtgaggggcccccccttatcccacctttccggctaggtgagggcgcgagcgggcgagcgagcgagagtggtgaggggggacggaaaagcagaattacctgtagctcttgttctgccatctcgggcgctctcacacaccttcacctgcacagacttgaaagtccagtttcaccagaggctgaggctccaggaaaagcggagcaagttcattggatcaaacatgtcacaagagtcggacaataataaaagactagtggccttagtgcccatgcccagtgaccctccattcaatacccgaagagcctacaccagtgaggatgaagcctggaagtcatacttggagaatcccctgacagcagccaccaaggccatgatgagcattaatggtgatgaggacagtgctgctgccctcggcctgctctatgactactacaaggttcctcgagacaagaggctgctgtctgtaagcaaagcaagtgacagccaagaagaccaggagaaaagaaactgccttggcaccagtgaagcccagagtaatttgagtggaggagaaaaccgagtgcaagtcctaaagactgttccagtgaacctttccctaaatcaagatcacctggagaattccaagcgggaacagtacagcatcagcttccccgagagctctgccatcatcccggtgtcgggaatcacggtggtgaaagctgaagatttcacaccagttttcatggccccacctgtgcactatccccggggagatggggaagagcaacgagtggttatctttgaacagactcagtatgacgtgccctcgctggccacccacagcgcctatctcaaagacgaccagcgcagcactccggacagcacatacagcgagagcttcaaggacgcagccacagagaaatttcggagtgcttcagttggggctgaggagtacatgtatgatcagacatcaagtggcacatttcagtacaccctggaagccaccaaatctctccgtcagaagcagggggagggccccatgacctacctcaacaaaggacagttctatgccataacactcagcgagaccggagacaacaaatgcttccgacaccccatcagcaaagtcaggagtgtggtgatggtggtcttcagtgaagacaaaaacagagatgaacagctcaaatactggaaatactggcactctcggcagcatacggcgaagcagagggtccttgacattgccgattacaaggagagctttaatacgattggaaacattgaagagattgcatataatgctgtttcctttacctgggacgtgaatgaagaggcgaagattttcatcaccgtgaattgcttgagcacagatttctcctcccaaaaaggggtgaaaggacttcctttgatgattcagattgacacatacagttataacaatcgtagcaataaacccattcatagagcttattgccagatcaaggtcttctgtgacaaaggagcagaaagaaaaatccgagatgaagagcggaagcagaacaggaagaaagggaaaggccaggcctcccaaactcaatgcaacagctcctctgatgggaagttggctgccatacctttacagaagaagagtgacatcacctacttcaaaaccatgcctgatctccactcacagccagttctcttcatacctgatgttcactttgcaaacctgcagaggaccggacaggtgtattacaacacggatgatgaacgagaaggtggcagtgtccttgttaaacggatgttccggcccatggaagaggagtttggtccagtgccttcaaagcagatgaaagaagaagggacaaagcgagtgctcttgtacgtgaggaaggagactgacgatgtgttcgatgcattgatgttgaagtctcccacagtgaagggcctgatggaagcgatatctgagaaatatgggctgcccgtggagaagatagcaaagctttacaagaaaagcaaaaaaggcatcttggtgaacatggatgacaacatcatcgagcactactcgaacgaggacaccttcatcctcaacatggagagcatggtggagggcttcaaggtcacgctcatggaaatctagccctgggtttggcatccgctttggctggagctctcagtgcgttcctccctgagagagacagaagccccagccccagaacctggagacccatctcccccatctcacaactgctgttacaagaccgtgctggggagtggggcaagggacaggccccactgtcggtgtgcttggcccatccactggcacctaccacggagctgaagcctgagcccctcaggaaggtgccttaggcctgttggattcctatttattgcccaccttttcctggagcccaggtccaggcccgccaggactctgcaggtcactgctagctccagatgagaccgtccagcgttcccccttcaagagaaacactcatcccgaacagcctaaaaaattcccatcccttctctctcacccctccatatctatctcccgagtggctggacaaaatgagctacgtctgggtgcagtagttataggtggggcaagaggtggatgcccactttctggtcagacacctttaggttgctctggggaaggctgtcttgctaaatacctccagggttcccagcaagtggccaccaggccttgtacaggaagacattcagtcaccgtgtaattagtaacacagaaagtctgcctgtctgcattgtacatagtgtttataatattgtaataatatattttacctgtggtatgtgggcatgtttactgccactggcctagaggagacacagacctggagaccgttttaatgggggtttttgcctctgtgcctgttcaagagacttgcagggctaggtagagggcctttgggatgttaaggtgactgcagctgatgccaagatggactctgcaatgggcatacctgggggctcgttccctgtccccagaggaagccccctctccttctccatgggcatgactctccttcgaggccaccacgtttatctcacaatgatgtgttttgcttgactttccctttgcgctgtctcgtgggaaaggtcattctgtctgagaccccagctccttctccagctttggctgcgggcatggcctgagctttctggagagcctctgcagggggtttgccatcagggccctgtggctgggtctgctgcagagctccttggctatcaggagaatcctggacactgtactgtgcctcccagtttacaaacacgcccttcatctcaagtggccctttaaaaggcctgctgccatgtgagagctgtgaacagctcagctctgagtcggcaggctggggcttcctcctgggccaccagatggaaagggggtattgtttgcctcactcctggatgctgcgttttaaggaagtgagtgagaaagaatgtgccaagatacctggctcctgtgaaaccagcctcaggagggaaactgggagagagaagctgtggtctcctgctacatgccctgggagctggaagagaaaaacactcccctaaacaatcgcaaaatgatgaaccatcatgggccactgttctctttgaggggacaggtttaggggtttgcgttcgcccttgtgggctgaagcactagctttttggtagctagacacatcctgcacccaaaggttctctacaaaggcccagatttgtttgtaaagcactttgactcttacctggaggcccgctctctaagggcttcctgcgctcccacctcatctgtccctgagatgcagagcaggatggagggtctgcttctagctcagctgtttctccttgaggttgcggaggaattgaattgaatgggacagagggcaggtgctgtggccaagaagatctccgagcagcagtgacggggcaccttgctgtgtgtcctctgggcatgttaacccttctgtggggccaaaggtttgcatcgtggatccagctgtgctccagtctgtcccctcctcctccactctgactgccacgccccggaccagcagcttggggaccctccagggtactaatggggctctgttctgagatggacaaattcagtgttggaaatacatgttgtactatgcacttcccatgctcctagggttaggaatagtttcaaacatgattggcagacataacaacggcaaatactcggactggggcataggactccagagtaggaaaaagacaaaagatttggcagcctgacacaggcaacctacccctctctctccagcctctttatgaaactgtttgtttgccagtcctgccctaaggcagaagatgaattgaagatgctgtgcatgtttcctaagtccttgagcaatcatggtggtgacaattgccacaagggatatgaggccagtgccaccagagggtggtgccaagtgccacatcccttccgatccattcccctctgcatcctcggagcaccccagtttgcctttgatgtgtccgctgtgtatgttagctgaactttgatgagcaaaatttcctgagcgaaacactccaaagagataggaaaacttgccgcctcttcttttttgtcccttaatcaaactcaaataagcttaaaaaaaatccatggaagatcatggacatgtgaaatgagcatttttttcttttttttttttaacaaagtctgaactgaacagaacaagactttttcctcatacatctccaaattgtttaaacttactttatgagtgtttgtttagaagttcggaccaacagaaaaatgcagtcagatgtcatcttggaattggtttctaaaagagtaaggcatgtccctgcccagaaacttaggaagcatgaaataaatcaaatgtttattttccttcttatttaaaatcatgcaaatgcaacagaaatagagggtttgtgccaaatgctatgaacggccctttcttaaagacaagcaagggagattgatatatgtacaatttgctctcatgttttaaaaaaaaaaaggtaaatgtaacttaatagttttgtaaatgggagagggggaatctataaactataaatacagttattttattttttgtacatttttaaggagaaaaaataaatattcataacataagagtaaaa

SEQ ID NO: 6 - protein, 625 aa, Homo sapiens
MSQESDNNKRLVALVPMPSDPPFNTRRAYTSEDEAWKSYLENPLTAATKAMMSINGDEDSAAALGLLYDYYKVPRDKRLLSVSKASDSQEDQEKRNCLGTSEAQSNLSGGENRVQVLKTVPVNLSLNQDHLENSKREQYSISFPESSAIIPVSGITVVKAEDFTPVFMAPPVHYPRGDGEEQRVVIFEQTQYDVPSLATHSAYLKDDQRSTPDSTYSESFKDAATEKFRSASVGAEEYMYDQTSSGTFQYTLEATKSLRQKQGEGPMTYLNKGQFYAITLSETGDNKCFRHPISKVRSVVMVVFSEDKNRDEQLKYWKYWHSRQHTAKQRVLDIADYKESFNTIGNIEEIAYNAVSFTWDVNEEAKIFITVNCLSTDFSSQKGVKGLPLMIQIDTYSYNNRSNKPIHRAYCQIKVFCDKGAERKIRDEERKQNRKKGKGQASQTQCNSSSDGKLAAIPLQKKSDITYFKTMPDLHSQPVLFIPDVHFANLQRTGQVYYNTDDEREGGSVLVKRMFRPMEEEFGPVPSKQMKEEGTKRVLLYVRKETDDVFDALMLKSPTVKGLMEAISEKYGLPVEKIAKLYKKSKKGILVNMDDNIIEHYSNEDTFILNMESMVEGFKVTLMEI

SEQ ID NO: 7 - DNA, 3033 bp, Homo sapiens
  CDS (180)..(935)
gaaggctgcctcgctggtccgaattcggtggcgccacgtccgcccgtctccgccttctgcatcgcggcttcggcggcttccacctagacacctaacagtcgcggagccggccgcgtcgtgagggggtcggcacggggagtcgggcggtcttgtgcatcttggctacctgtgggtcgaagatgtcggacatcggagactggttcaggagcatcccggcgatcacgcgctattggttcgccgccaccgtcgccgtgcccttggtcggcaaactcggcctcatcagcccggcctacctcttcctctggcccgaagccttcctttatcgctttcagatttggaggccaatcactgccaccttttatttccctgtgggtccaggaactggatttctttatttggtcaatttatatttcttatatcagtattctacgcgacttgaaacaggagcttttgatgggaggccagcagactatttattcatgctcctctttaactggatttgcatcgtgattactggcttagcaatggatatgcagttgctgatgattcctctgatcatgtcagtactttatgtctgggcccagctgaacagagacatgattgtatcattttggtttggaacacgatttaaggcctgctatttaccctgggttatccttggattcaactatatcatcggaggctcggtaatcaatgagcttattggaaatctggttggacatctttattttttcctaatgttcagatacccaatggacttgggaggaagaaattttctatccacacctcagtttttgtaccgctggctgcccagtaggagaggaggagtatcaggatttggtgtgccccctgctagcatgaggcgagctgctgatcagaatggcggaggcgggagacacaactggggccagggctttcgacttggagaccagtgaaggggcggcctcgggcagccgctcctctcaagccacatttcctcccagtgctgggtgcacttaacaactgcgttctggctaacactgttggacctgacccacactgaatgtagtctttcagtacgagacaaagtttcttaaatcccgaagaaaaatataagtgttccacaagtttcacgattctcattcaagtccttactgctgtgaagaacaaataccaactgtgcaaattgcaaaactgactacattttttggtgtcttctcttctcccctttccgtctgaataatgggttttagcgggtcctaatctgctggcattgagctggggctgggtcaccaaacccttcccaaaaggaccttatctctttcttgcacacatgcctctctcccacttttcccaacccccacatttgcaactagaaaaagttgcccataaaattgctctgcccttgacaggttctgttatttattgacttttgccaaggctggtcacaacaatcatattcacgttattttccccttttggtggcagaactgttaccaatagggggagaagacagccacggatgaagcgtttctcagcttttggaattgcttcgactgacatccgttgttaaccgtttgccactcttcagatattttttataaaaaaagtaccactgagttcatgagggccacagattggttattaatgagatacgagggttggtgctgggtgtttgtttcctgagctaagtgatcaagactgtagtggagttgcagctaacatgggttaggtttaaaccatgggggatgcacccctttgcgtttcatatgtagccctactggctttgtgtagctggagtagttgggttgctttgtgttaggaggatccagatcatgttggctacagggagatgctctctttgagaggtcctgggcattgattcccatttcaatctcattctggatatgtgttcattgagtaaaggaggagagaccctcatacgctatttaaatgtcacttttttgcctatcccccgttttttggtcatgtttcaattaattgtgaggaaggcgcagctcctctctgcacgtagatcattttttaaagctaatgtaagcacatctaagggaataacatgatttaaggttgaaatggctttagaatcatttgggtttgagggtgtgttattttgagtcatgaatgtacaagctctgtgaatcagaccagcttaaatacccacacctttttttcgtaggtgggcttttcctatcagagcttggctcataaccaaataaagttttttgaaggccatggcttttcacacagttattttattttatgacgttatctgaaagcagactgttaggagcagtattgagtggctgtcacactttgaggcaactaaaaaggcttcaaacgttttgatcagtttcttttcaggaaacattgtgctctaacagtatgactattctttcccccactcttaaacagtgtgatgtgtgttatcctaggaaatgagagttggcaaacaacttctcattttgaatagagtttgtgtgtacttctccatatttaatttatatgataaaataggtggggagagtctgaaccttaactgtcatgttttgttgttcatctgtggccacaataaagtttacttgtaaaattttagaggccattactccaattatgttgcacgtacactcattgtacaggcgtggagactcattgtatgtataagaatatttctgacagtgagtgacccggagtctctggtgtaccctcttaccagtcagctgcctgcgagcagtcattttttcctaaaggtttacaagtatttagaacttttcagttcagggcaaaatgttcatgaagttattcctcttaaacatggttaggaagctgatgacgttattgattttgtctggattatgtttctggaataattttaccaaaacaagctatttgagttttgacttgacaaggcaaaacatgacagtggattctctttacaaatggaaaaaaaaaatccttattttgtataaaggacttccctttttgtaaactaatcctttttattggtaaaaattgtaaattaaaatgtgcaacttg

SEQ ID NO: 8 - protein, 251 aa, Homo sapiens
MSDIGDWFRSIPAITRYWFAATVAVPLVGKLGLISPAYLFLWPEAFLYRFQIWRPITATFYFPVGPGTGFLYLVNLYFLYQYSTRLETGAFDGRPADYLFMLLFNWICIVITGLAMDMQLLMIPLIMSVLYVWAQLNRDMIVSFWFGTRFKACYLPWVILGFNYIIGGSVINELIGNLVGHLYFFLMFRYPMDLGGRNFLSTPQFLYRWLPSRRGGVSGFGVPPASMRRAADQNGGGGRHNWGQGFRLGDQ

SEQ ID NO: 9 - DNA, 21 bp, Artificial sequence
  Survivin _ Forward
ccagatgacgaccccatagag

SEQ ID NO: 10 - DNA, 23 bp, Artificial sequence
  Survivin _ Reverse
ttgttggtttcctttgcaatttt

SEQ ID NO: 11 - DNA, 22 bp, Artificial sequence
  Survivin _ Probe
cattcgtccggttgcgctttcc

SEQ ID NO: 12 - DNA, 20 bp, Artificial sequence
  Survivin-2B_ Forward
aagaactggcccttcttgga

SEQ ID NO: 13 - DNA, 24 bp, Artificial sequence
  Survivin-2B _ Reverse
ccaagtgctggtattacaggcgta

SEQ ID NO: 14 - DNA, 23 bp, Artificial sequence
  Survivin-2B _ Probe
actgccccactgagaacgagcca

SEQ ID NO: 15 - DNA, 22 bp, Artificial sequence
  Survivin- delta Ex3 _ Forward
cccagtgtttcttctgcttcaa

SEQ ID NO: 16 - DNA, 24 bp, Artificial sequence
  Survivin- delta Ex3 _ Reverse
ttcttcgcagtttcctcaaattct

SEQ ID NO: 17 - DNA, 26 bp, Artificial sequence
  Survivin- delta Ex3 _ Probe
acgaccccatgcaaaggaaaccaaca

SEQ ID NO: 18 - DNA, 21 bp, Artificial sequence
  Survivin-3B _ Forward
ccagatgacgaccccatagag

SEQ ID NO: 19 - DNA, 20 bp, Artificial sequence
  Survivin-3B _ Reverse
aagaactggcccttcttgga

SEQ ID NO: 20 - DNA, 22 bp, Artificial sequence
  Survivin-3B _ Probe
cattcgtccggttgcgctttcc

SEQ ID NO: 21 - DNA, 25 bp, Artificial sequence
  Survivin-2 alpha _ Forward
gctttgttttgaactgagttgtcaa

SEQ ID NO: 22 - DNA, 19 bp, Artificial sequence
  Survivin-2 alpha _ Reverse
gcaatgagggtggaaagca

SEQ ID NO: 23 - DNA, 35 bp, Artificial sequence
  Survivin-2 alpha _ Probe
agatttgagttgcaaagacacttagtatgggaggg

SEQ ID NO: 24 - DNA, 21 bp, Artificial sequence
  Caspase-3 _ Forward
ctggactgtggcattgagaca

SEQ ID NO: 25 - DNA, 20 bp, Artificial sequence
  Caspase-3 _ Reverse
agtcggcctccatggtattt

SEQ ID NO: 26 - DNA, 26 bp, Artificial sequence
  Caspase-3 _ Probe
tggtgttgatgatgacatggcgtgtc

SEQ ID NO: 27 - DNA, 26 bp, Artificial sequence
  Caspase-3s _ Forward
agaagtctaactggaaaacccaaact

SEQ ID NO: 28 - DNA, 20 bp, Artificial sequence
  Caspase-3s _ Reverse
caaagcgactggatgaacca

SEQ ID NO: 29 - DNA, 25 bp, Artificial sequence
  Caspase-3s _ Probe
attattcaggttattattcttggcg

(19) United States
(12) Patent Application Publication
(10) Pub. No.: US 2011/0189185 A1
(43) Pub. Date: Aug. 4, 2011

(54) Method for Predicting Responsiveness to a Treatment With an Anti-HER2 Antibody

(75) Inventors: Sarab Lizard, Fontaine Les Dijon (FR); Romain Boidot, Sint-Stevens-Woluwe (BE); Laurent Arnould, Dijon (FR); Bruno Coudert, Dijon (FR); Pierre Fumoleau, Fontaine Les Dijon (FR)

(73) Assignee: Centre Georges Francois Leclerc, Dijon Cedex (FR)

(21) Appl. No.: 12/994,989

(22) PCT Filed: Jun. 8, 2009
(86) PCT No.: PCT/EP2009/057036

§ 371 Date: Apr. 21, 2011

(30) Foreign Application Priority Data
Jun. 9, 2008 (EP) 08305241.5

(51) Int. Cl. A61K 39/395; C40B 40/08; C12Q 1/68; C40B 30/00; A61P 35/00; G01N 33/53

(52) U.S. Cl. 424/138.1; 506/17; 435/6.12; 506/7; 435/6.1; 435/7.1

(57) ABSTRACT

The invention provides an in vitro method for predicting whether a patient would be responsive to a treatment with an anti-HER2 blocking agent, such as trastuzumab, which method comprises determining the expression level of at least 4 genes in a biological sample of said patient, wherein said genes are GPR22, PEX19, GRHL2 and DERL1. The invention further provides a DNA chip for performing such method.

The present invention relates to a method for predicting the response to a treatment with a HER2 blocking agent, such as an anti-HER2 antibody.

The human epidermal growth factor receptor 2 (HER2) proto-oncogene encodes a 185 kDa glycoprotein receptor tyrosine kinase, which is a member of the growth factor receptor family that includes epidermal growth factor receptor (EGFR) 1, 3, and 4. Amplification and overexpression of HER2 is observed in 20-30% of invasive breast cancer and correlates with tumor progression and poor prognosis. Although the EGFR family stimulates mitogenesis through ligand-induced pathways, there is no known ligand for HER2. Increased HER2 expression induces a signaling pathway that involves Ras and Src as well as PI3K/Akt and is associated with tumor formation. The transforming ability of HER2 has been linked to cell survival and through mitogenic signaling pathways.

Trastuzumab (Herceptin®; F. Hoffmann-La Roche, Basel, Switzerland) is a humanized monoclonal antibody directed against the HER2 protein. It produces significant (>50%) tumor regression in ˜15% of patients with HER2-positive metastatic breast cancer that is refractory to conventional therapy, and in ˜23% of patients when used as first-line therapy (Cobleigh et al, 1999). The addition of trastuzumab to standard chemotherapy significantly improves response rate, response duration, and survival. The clinical benefits of trastuzumab-based therapies have been well documented in both the adjuvant and the metastatic settings.

However resistance to trastuzumab is often observed in women with HER2-positive breast cancer and has been shown to involve multiple potential mechanisms.

The precise molecular pathways through which trastuzumab exerts its antitumor effects in breast cancer cells, or through which a patient shows resistance to these antitumor effects, are not yet fully understood.

Trastuzumab action involves multiple mechanisms including the induction of apoptotic signaling pathways, cell cycle perturbation, cellular cytotoxicity, and inhibition of nuclear excision repair mechanisms. Treatment with trastuzumab dephosphorylates and down-regulates HER2, leading to significant clinical efficacy against HER2-positive breast cancer. It also sensitizes breast cancer cells to chemotherapeutic agents, especially tubulin-polymerizing agents and radiation therapy. It was demonstrated that anti-HER2 monoclonal antibodies inhibit HER2-overexpressing breast cancer cells through G1 cell cycle arrest that was associated with the induction of the cyclin-dependent kinase (CDK) inhibitor p27kip1 and reduction of CDK2. Trastuzumab may also inhibit the PI3K/Akt pathway by promoting PTEN activation. Overexpression of HER2 in human tumor cells has also been shown to be associated with increased angiogenesis and expression of vascular endothelial growth factor, and trastuzumab has been shown to reduce tumor volume and microvessel density in HER2-positive breast cancer models in vivo. Synergy with DNA-damaging drugs is thought to be due to trastuzumab-mediated inhibition of DNA repair. Trastuzumab partially inhibits repair of DNA adducts in vitro after treatment with cisplatin and blocks unscheduled DNA synthesis after radiation. Finally, trastuzumab has also been shown to be associated with immunoreactive actions via antibody-directed cellular cytotoxicity (ADCC).

Recently, trastuzumab-based neoadjuvant chemotherapy has been shown to achieve promising efficacy, with a good pathological complete response (pCR) rate, while being well tolerated in women with stage II or III HER2-positive breast cancer (Buzdar et al, 2005; Coudert et al, 2006; Coudert et al, 2007). Among taxanes, docetaxel associated with trastuzumab shows evidence of improved efficacy in obtaining pCR rates.

To date, only one study has used RNA profiling to predict responses to trastuzumab-vinorelbine-based treatments in patients with early HER2-positive breast cancer (Harris et al, 2007). In this study, resistant tumors exhibited a higher expression of several growth factors, growth factor receptors, the PI3K regulatory subunit p85, microtubule-associated protein 2, and some basal genes.

There is still a need for a complete molecular signature which would be useful for predicting responsiveness to an anti-HER2 antibody treatment, especially a trastuzumab-docetaxel-based chemotherapy.

### SUMMARY OF THE INVENTION

Using microarray analysis, the inventors performed a RNA profiling and found a signature of pathological complete response (pCR).

On this basis the invention provides an in vitro method for predicting whether a patient would be responsive to a treatment with a HER2 blocking agent, preferably an anti-HER2 antibody such as trastuzumab, which method comprises determining the expression level of at least 4 genes in a biological sample of said patient, wherein said genes are GPR22, PEX19, GRHL2 and DERL1.

In a particular embodiment, the invention provides an in vitro method for predicting whether a patient would be responsive to a treatment with a combination of trastuzumab and docetaxel, which method comprises determining the expression level of at least 4 genes in a biological sample of said patient, wherein said genes are GPR22, PEX19, GRHL2 and DERL1.

The combined expression profile of these genes is informative of the status of the patient who, before any treatment with a HER2 blocking agent, can be classified as responder or non-responder, and be given the appropriate treatment.

The method usually comprises the further step of comparing the expression level of said genes with reference values obtained from responder and non-responder groups of patients.

Generally speaking, the patient is preferably affected with a HER2-positive cancer. The patient is preferably with breast cancer.

The expression level is advantageously determined by quantifying the level of mRNA of said genes in the biological sample. Using a DNA chip is particularly useful in that respect. The assay using such a chip is indeed reliable, fast, and cheap.

A further subject of the invention is the DNA chip that allows performing such method.

### DETAILED DESCRIPTION OF THE INVENTION

The inventors examined the expression of a panel of genes involved in cell cycle progression, DNA repair, and apoptosis that may have a putative role in trastuzumab resistance, in a series of breast carcinomas that had been treated with trastuzumab-based neoadjuvant chemotherapy. In parallel, they used microarray analysis on the same tumor samples in order to identify a potential marker of pCR that may have prognostic value in the identifying patients who are more likely to respond to trastuzumab therapy.

On this basis, the inventors identified a set of genes whose combined expression profiles allow to distinguish patients between responder and non-responder to a treatment with a HER2 blocking agent, preferably an anti-HER2 antibody such as trastuzumab.

In practice, the rapid determination of the expression level of said genes, e.g. by a quantitative RT-PCR, offers a powerful tool for predicting responsiveness to a HER2 blocking agent, preferably an anti-HER2 antibody such as trastuzumab.

The study presented in the Examples shows that such analysis allows to predict efficacy of a HER2 blocking agent, preferably an anti-HER2 antibody such as trastuzumab, with a sensitivity of 100%, a specificity of 78%, and a positive predictive value of 67%, and a negative predictive value of 100%.

### Patients

The term “patient” refers to any subject (preferably human) afflicted with a disease likely to benefit from a treatment with a HER2 blocking agent, in particular a HER2-related disease. The patient may be a man or a woman, preferably a woman, especially a woman with breast cancer.

Such diseases include benign and malignant tumors; leukemias and lymphoid malignancies; neuronal, glial, astrocytal, hypothalamic and other glandular, macrophagal, epithelial, stromal and blastocoelic disorders; and inflammatory, angiogenic and immunologic disorders. Cancers are more preferably targeted.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, liver cancer, prostate cancer, vulval cancer, thyroid cancer, hepatic carcinoma and various types of head and neck cancer.

The patient is preferably affected with a HER2-positive disease, in particular a HER2-positive cancer.

Preferably the HER2 positive cancer is primary tumor. Alternatively the HER2 positive cancer is a secondary tumor such as e.g. a locally advanced cancer or a metastatic cancer.

The term “HER2-positive” means that the HER2 protein is overexpressed, i.e. shows an abnormal level of expression in a cell from a disease within a specific tissue or organ of the patient relative to the level of expression in a normal cell from that tissue or organ. Patients having a cancer characterized by overexpression of the HER2 receptor can be determined by standard assays known in the art. Preferably overexpression is measured in fixed cells of frozen or paraffin-embedded tissue sections using immunohistochemical (IHC) detection. When coupled with histological staining, localization of the targeted protein can be determined and extent of its expression within a tumor can be measured both qualitatively and semi-quantitatively. Such IHC detection assays are known in the art and include the Clinical Trial Assay (CTA), the commercially available LabCorp® 4D5 test, and the commercially available DAKO HercepTest® (DAKO, Carpinteria, Calif.). The latter assay uses a specific range of 0 to 3+ cell staining (0 being normal expression, 3+ indicating the strongest positive expression) to identify cancers having overexpression of the HER2 protein. Thus, patients having a cancer characterized by overexpression of the HER2 protein in the range of 1+, 2+, or 3+, preferably 2+ or 3+, more preferably 3+ are particularly encompassed.

Using standard detection assays, several types of cancers have been characterized as having cells that overexpress the HER2 protein. Such cancers include, but are not limited to, breast, gastric, endometrial, salivary gland, non-small cell lung, pancreatic, renal, ovarian, peritoneal, prostate, bladder, colorectal cancers, and glioblastomas. Methods of the invention are useful in the treatment/management of any such cancer whose cells overexpress the HER2 protein. Of particular interest is breast cancer.

A “responder” patient, or group of patients, refers to a patient, or group of patients, who shows or will show a clinically significant relief in the disease when treated with a HER2 blocking agent.

The expression level of the genes or transcripts is determined and compared between a responder group and a non-responder group of patients. Said “expression level of genes or transcripts” corresponds to the combined expression profile of said genes or transcripts in either group.

The comparison between groups can be performed by computer tools, using the Mann and Whitney test performed with 1000 permutations (Zoe software, IGBM Strasbourg). These tools take into account the differential expression of the gene clusters, i.e. of the combination of the genes between groups, and generate an algorithm. The latter next allows for a prediction of the non-responder or responder status of any further patient, provided the same transcript level has been determined in said patient.

After being tested for responsiveness to a treatment with a HER2 blocking agent, the patients may be prescribed with a HER2 blocking agent with or without the same basic treatment.

While the methods of the invention are directed to treatment of an existing disease, such as cancer, it is recognized that the methods may be useful in preventing further disease development, including tumor outgrowths arising during therapy. The methods of the invention are particularly useful in the treatment of subjects having breast cancer, more particularly subjects having metastatic breast cancer and experiencing a relapse following one or more chemotherapy regimens for their metastatic disease.

### Chemotherapy Choice

The method of the invention makes it possible to discriminate between “responder” and “non-responder” patients to a treatment with a HER2 blocking agent

“HER2 blocking agents” refer to molecules, such as proteins or small molecules, that can significantly reduce HER2 properties.

Such blocking agents include anti-HER2 antibodies, e.g. trastuzumab, pertuzumab, or cetuximab. Preferably the anti-HER2 antibody is trastuzumab.

Trastuzumab (sold under the tradename Herceptin®) is a recombinant humanized anti-HER2 monoclonal antibody used for the treatment of HER2 over-expressed/HER2 gene amplified metastatic breast cancer. Trastuzumab binds specifically to the same epitope of HER2 as the murine anti-HER2 antibody 4D5. Trastuzumab is a recombinant humanized version of the murine anti-HER2 antibody 4D5, referred to as rhuMAb 4D5 or trastuzumab) and has been clinically active in patients with HER2-overexpressing metastatic breast cancers that had received extensive prior anticancer therapy. Trastuzumab and its method of preparation are described in U.S. Pat. No. 5,821,337.

In a preferred embodiment, the method of the invention is useful for predicting whether a patient would be responsive to a treatment with a HER2 blocking agent combined with a taxane, such as docetaxel.

“Docetaxel” refers to the active ingredient of Taxotere®.

### The Sets of Predictive Genes or Transcripts

All the genes or transcripts identified are known per se, and listed in the below tables A, B and C.

Table A presents the set of four genes whose combined expression profile has been shown to be informative with regard to responsiveness to a treatment with HER2 blocking agent. These are the GPR22, PEX19, GRHL2 and DERL1 transcripts.

TABLE A
subset of 4 genes (transcripts)

| | GENBANK access | | |
|---|---|---|---|
| Gene | number | Name | Seq ID NO: |
| GPR22 | NM_005295 | G protein-coupled | 1 |
| | | receptor 22 | |
| PEX19 | NM_002857 | Peroxisomal | 3 |
| | | biogenesis factor 19 | |
| GRHL2 | NM_024915 | Grainyhead-like 2 | 5 |
| | | (Drosophila) | |
| DERL1 | NM_024295 | Derlin 1 | 7 |

In the responder group, GPR22 and GRHL2 are overexpressed while PEX19 and DER1 are overexpressed in the non-responder group.

In a particular embodiment, the method of the invention further comprises determining the expression level of the genes or transcripts of Table B, or of a subcombination thereof (combined with the set of four genes or transcripts as defined in Table A):

TABLE B
Other transcripts of interest for the predictive method

| AgilentSpotID | GenbankID | UGCluster | Name | Symbol | p value |
|---|---|---|---|---|---|
| as00595 | AK095652 | Hs.494822 | Hypothetical protein LOC158402 | LOC158402 | 0 |
| as02991 | NM_003390 | Hs.249441 | WEE1 homolog (S. pombe) | WEE1 | 0.001 |
| as03022 | AL117644 | Hs.429819 | Phosphatidylinositol transfer protein, alpha | PITPNA | 0.001 |
| as04760 | AK022035 | Hs.659665 | CDNA FLJ11973 fis, clone HEMBB1001221 | | 0 |
| as05104 | NM_002715 | Hs.483408 | Protein phosphatase 2 (formerly 2A), catalytic subunit, alpha isoform | PPP2CA | 0.001 |
| as06024 | NM_007145 | Hs.643436 | Zinc finger protein 146 | ZNF146 | 0.001 |
| as06108 | NM_020654 | Hs.529551 | SUMO1/sentrin specific peptidase 7 | SENP7 | 0 |
| as06408 | NM_080670 | Hs.406840 | Solute carrier family 35, member A4 | SLC35A4 | 0.001 |
| as06448 | XM_045127 | Hs.605380 | Homo sapiens KIAA1549 protein | KIAA1549 | 0 |
| as08524 | NM_003204 | Hs.514284 | Nuclear factor (erythroid-derived 2)-like 1 | NFE2L1 | 0.001 |
| as09257 | NM_005295 | Hs.657277 | G protein-coupled receptor 22 | GPR22 | 0 |
| as10291 | NM_006372 | Hs.571177 | Synaptotagmin binding, cytoplasmic RNA interacting protein | SYNCRIP | 0 |
| as11424 | NM_018691 | Hs.166551 | Chromosome 5 open reading frame 3 | C5orf3 | 0.001 |
| as11638 | NM_002857 | Hs.517232 | Peroxisomal biogenesis factor 19 | PEX19 | 0 |
| as12494 | NM_002558 | Hs.41735 | Purinergic receptor P2X, ligand-gated ion channel, 1 | P2RX1 | 0.001 |
| as15670 | NM_017964 | Hs.23248 | Solute carrier family 30 (zinc transporter), member 6 | SLC30A6 | 0 |
| as15820 | NM_003672 | Hs.127411 | CDC14 cell division cycle 14 homolog A (S. cerevisiae) | CDC14A | 0.001 |
| as16749 | NM_024915 | Hs.661088 | Grainyhead-like 2 (Drosophila) | GRHL2 | 0 |
| as18913 | NM_145204 | Hs.513002 | SUMO/sentrin specific peptidase family member 8 | SENP8 | 0.001 |
| as19535 | NM_002815 | Hs.655396 | Proteasome (prosome, macropain) 26S subunit, non-ATPase, 11 | PSMD11 | 0.001 |
| as20561 | NM_004937 | Hs.187667 | Cystinosis, nephropathic | CTNS | 0 |
| as21050 | NM_024295 | KIAA1549 | Derlin 1 | DERL1 | 0 |
| as21549 | NM_032816 | Hs.599703 | Coiled-coil domain containing 123 | CCDC123 | 0 |
| as21885 | NM_002730 | Hs.631630 | Protein kinase, cAMP-dependent, catalytic, alpha | PRKACA | 0.001 |
| as22719 | NM_000227 | Hs.436367 | Laminin, alpha 3 | LAMA3 | 0.001 |
| as22767 | NM_017694 | Hs.644886 | FLJ20160 protein | FLJ20160 | 0.001 |
| as24280 | XM_295178 | Not available | Homo sapiens hypothetical LOC340171 | LOC340171 | 0 |
| as25304 | NM_004603 | Hs.647024 | Syntaxin 1A (brain) | STX1A | 0.001 |

TABLE C
Subgroup of transcripts of interest for the predictive method

| Gene | Genbank ID | Description |
|---|---|---|
| LOC158402 | AK095652 | Hypothetical protein LOC158402 |
| | AK022035 | CDNA FLJ11973 fis, clone HEMBB1001221 |
| SENP7 | NM_020654 | SUMO1/sentrin specific peptidase 7 |
| KIAA1549 | XM_045127 | Homo sapiens KIAA1549 protein |
| GPR22 | NM_005295 | G protein-coupled receptor 22 |
| SYNCRIP | NM_006372 | Synaptotagmin binding, cytoplasmic RNA interacting protein |
| PEX19 | NM_002857 | Peroxisomal biogenesis factor 19 |
| SLC30A6 | NM_017964 | Solute carrier family 30 (zinc transporter), member 6 |
| GRHL2 | NM_024915 | Grainyhead-like 2 (Drosophila) |
| CTNS | NM_004937 | Cystinosis, nephropathic |
| DERL1 | NM_024295 | Derlin 1 |
| CCDC123 | NM_032816 | Coiled-coil domain containing 123 |
| LOC340171 | XM_295178 | Homo sapiens hypothetical LOC340171 |

Among the 28 genes of Table B, 12 were shown to be more highly expressed in pCR tumor samples (WEE1, ZNF146, SENP7, GPR22, SYNCRIP, SLC30A6, GRHL2, CCDC123, STX1A, cDNA FLJ11973 fis, clone HEMBB1001221, KIAA1549 and Homo sapiens hypothetical LOC340171), and 16 genes were shown to be highly expressed in non-pCR samples (LOC158402, PITPNA, PPP2CA, SLC35A4, NFE2L1, C5orf3, PEX19, P2RX1, CDC14A, SENP8, PSMD11, CTNS, PRKACA, LAMA3, FLJ20160, and DERL1).

### Determination of Expression Level

Determination of the expression level of a gene or transcript can be performed by a variety of techniques, from a biological sample. The term “biological sample” means any biological sample derived from a patient, preferably a sample which contains nucleic acids. Examples of such samples include fluids, tissues, cell samples, organs, biopsies, etc. Most preferred samples are disease tissue samples. Blood, plasma, saliva, urine, seminal fluid, etc, may also be used. The biological sample may be treated prior to its use, e.g. in order to render nucleic acids available. Techniques of cell or protein lysis, concentration or dilution of nucleic acids, are known by the skilled person.

Generally, the expression level as determined is a relative expression level.

More preferably, the determination comprises contacting the sample with selective reagents such as probes, primers or ligands, and thereby detecting the presence, or measuring the amount, of polypeptide or nucleic acids of interest originally in the sample. Contacting may be performed in any suitable device, such as a plate, microtiter dish, test tube, well, glass, column, and so forth In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a preferred embodiment, the expression level may be determined by determining the quantity of mRNA.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (a g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used herein may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, et 2006)

In this context, the invention further provides a DNA chip comprising a solid support which carries nucleic acids that are specific to GPR22, PEX19, GRHL2 and DERL1 genes.

In a particular embodiment, the invention provides a DNA chip which further carries nucleic acids that are specific to any or all of the transcripts listed in Table B, or a subcombination thereof.

In a preferred embodiment, the invention more particularly provides a DNA chip comprising a solid support which carries nucleic acids that are specific to GPR22, PEX19, GRHL2 and DERL1 genes, and which further carries nucleic acids that are specific to any or all of the transcripts listed in Table C.

Other methods for determining the expression level of said genes include the determination of the quantity of proteins encoded by said genes.

Such methods comprise contacting a biological sample with a binding partner capable of selectively interacting with a marker protein present in the sample. The binding partner is generally an antibody, that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Preferably, the expression level is compared to a reference expression level, for instance the expression level of the genes in cell-lines or responder patients. The method can comprise the step of comparing the expression levels of the genes determined in the sample to reference or control expression levels. In an embodiment, the reference or control expression levels are determined with a sample of cells, preferably cancer cells, which are sensitive to an anti-HER2 antibody. Alternatively, reference or control expression levels are determined with a sample of patients or subjects insensitive to the treatment with an anti-HER2 antibody. However, the man skilled in art understands that other references can be used. For instance, the invention also contemplates a reference level corresponding to the expression level in a cell resistant to an anti-HER2 antibody. The method can also comprise the determination of the expression level for control genes. The control genes are chosen among the genes known to have a constant expression level, in particular between sensitive and resistant cells to an anti-HER2 antibody. In addition, the expression level of at least one control gene is determined in order to normalize the result.

### Therapeutic Applications

A method for treating a HER2-related disease is contemplated, which method comprises
a) a preliminary step of testing whether a patient with a HER2-related disease would be responsive to a treatment with a HER2 blocking agent, by determining the expression level of four genes in a biological sample of said patient, wherein said genes are GPR22, PEX19, GRHL2 and DERL1; hereby classifying the patient as responder or non responder;
b) a step of administering a HER2 blocking agent to a patient classified as responder.

The classification of the patient—as responder or non-responder—is made according to the combined expression level of said four genes. It allows to define a subgroup of patients who will be responsive to a treatment with a HER2 blocking agent.

Another particular subject of the invention is thus a HER2 blocking agent, such as an anti-HER2 antibody, for treating a patient with a HER2-related disease, such as a cancer, and classified as responder to a treatment with a HER2 blocking agent, by the method described above.

The example illustrates the invention without limiting its scope.

### Example 1

### Gene Expression Profile and Response to Trastuzumab-Docetaxel-Based Treatment in Breast Carcinoma

Materials and Methods

Patients and Samples

The inventors retrospectively studied a population of 38 patients who had received trastuzumab in combination with chemotherapy as primary systemic therapy for their operable, HER2-positive, stage II/III breast cancer (Table 1).

TABLE 1
Demographic data

| | Training set | Independent set | Total |
|---|---|---|---|
| | (n = 25) | (n = 13) | (n = 38) |
| Age (years) | | | |
| ≦50 | 13 | 9 | 22 |
| >50 | 12 | 4 | 16 |
| SBR grade | | | |
| I | 1 | 0 | 1 |
| II | 14 | 8 | 22 |
| III | 10 | 4 | 14 |
| Unknown | 0 | 1 | 1 |
| Hormone receptors | | | |
| ER-negative | 13 | 3 | 16 |
| ER-positive | 12 | 10 | 22 |
| PR-negative | 16 | 3 | 19 |
| PR-positive | 9 | 10 | 19 |
| Tumor size (cm) | | | |
| <2 | 1 | 0 | 1 |
| 2-4 | 17 | 12 | 29 |
| >4 | 6 | 1 | 7 |
| ND | 1 | 0 | 1 |
| Treatment | | | |
| TH | 18 | 11 | 29 |
| TCH | 7 | 2 | 9 |
| Pathological response | | | |
| pCR | 11 | 4 | 15 |
| non-pCR | 14 | 9 | 23 |

ER, estrogen receptor;
non-pCR, absence of pathological complete response;
ND, not determined;
pCR, pathological complete response;
PR, progesterone receptor
SBR, Scarff-Bloom-Richardson;
TCH, trastuzumab + carboplatin + docetaxel;
TH, trastuzumab + docetaxel

All patients provided written, informed consent for their tissue material and clinical data to be used for research purposes. Patients were treated in two open-label phase II clinical trials: TAXHER01 (n=29) and GETNA01 (n=10) (Coudert et al, 2006; Coudert et al, 2007).

All patients received weekly neoadjuvant trastuzumab (4 mg/kg loading dose followed by 2 mg/kg once weekly) in combination with either docetaxel alone (100 mg/m2 every 3 weeks for six cycles) or docetaxel (75 mg/m2 every 3 weeks for six cycles) combined with carboplatin (AUC 6) every 3 weeks for six cycles. pCR rates were assessed using Chevallier's classification (Chevallier et al, 1993) 3 weeks after the last course of trastuzumab-containing neoadjuvant treatment. An absence of disease in the breast or in the lymph nodes, with or without in situ carcinoma, was considered to be a pCR. HER2 status was determined using both immunohistochemistry and fluorescence in situ hybridization (Arnould et al, 2007).

Needle core biopsies were taken at baseline, with one used for the initial diagnosis and two used for RNA extraction. All tissue samples were snap frozen and stored in liquid nitrogen, and only samples containing ≧30% tumor cells were analyzed further.

RNA Extraction

Total RNA was extracted from tissue samples by using the TRIzol® method as recommended by the manufacturer (Invitrogen Corporation, Carlsbad, Calif., USA). The quantity and purity of the extracted RNA were assessed using a NanoDrop® 1000 spectrophotometer (NanoDrop, Wilmington, Del., USA) at 260 and 280 nm (the A260/280 ratio of pure RNA is higher than 1.8). The quality of the extracted RNA was determined using an Agilent 2100 bioanalyzer (Agilent, Santa Clara, Calif., USA). Total RNA from a pool of four normal mammary tissues was used as a normal sample, and RNA extracted from the MCF-7 human breast cancer cell line was used to calibrate real-time quantitative and reverse transcriptase polymerase chain reaction (RT-PCR).

RT-PCR and Real-Time Quantitative PCR

One microgram of total RNA was reverse transcribed in 20 μl of RT-PCR (Arnal et al, 2000). The real-time quantitative PCR was performed on ABI PRISM® 7300 (Applied Biosystems, Foster City, Calif., USA) using the TaqMan® method. Analysis of 18S ribosomal RNA was used to assess complementary DNA (cDNA) quality and as a reference control. Results were analyzed at the Ct level and references for the genes analyzed are summarized in Table 2.

TABLE 2
References and nucleotide sequences of
primers and probes used in this study

| | Gene or | Reference | |
|---|---|---|---|
| Function | transcript | NCBI | Reference or sequences |
| | cdc27 | NM_001256 | Hs01559427_m1 |
| | SKP2 | NM_032637 | Hs01021867_m1 |
| | p27 | NM_004064 | Hs00153277_ml |
| | p53 | NM_000546 | Hs00153340_ml |
| Cell cycle | c-Myc | NM_002467 | Hs00153408_ml |
| | Cyclin B2 | NM_004701 | Hs00270424_ml |
| | RBX1 | NM_014248 | Hs00360274_m1 |
| | CCL4 | NM_002984 | Hs99999148_ml |
| | CDC451 | NM_003504 | Hs00907337_m1 |
| DNA repair | XRCC2 | NM_005431 | Hs00538799_ml |
| | ERCC2 | NM_000400 | Hs00361161_m1 |
| | MREIIA | NM_005591 | Hs00967442_m1 |
| | HMOX2 | NM_002134 | Hs01558390_m1 |
| | MSH5 | NM_002441 | Hs00159268_m1 |
| Apoptosis | Survivin | NM_001168 | F: 5'-ccagatgacgaccccatagag-3' |
| | | | (SEQ ID NO: 9) |
| | | | R: 5'-ttgttggtttcctttgcaatttt-3' |
| | | | (SEQ ID NO:10) |
| | | | P: 5'-cattcgtccggttgcgctttcc-3' |
| | | | (SEQ ID NO: 11) |
| | Survivin-2B | NM_001012271 | F: 5'-aagaactggccatcttgga-3' |
| | | | (SEQ ID NO:12) |
| | | | R: 5'-ccaagtgctggtattacaggcgta-3' |
| | | | (SEQ ID NO: 13) |
| | | | P: 5'-actgccccactgagaacgagcca-3' |
| | | | (SEQ ID NO: 14) |
| | Survivin-ΔEx3 | NM_001012270 | F: 5'-cccagtgtttcttctgcttcaa-3' |
| | | | (SEQ ID NO: 15) |
| | | | R: 5'-ttatcgcagtttcctcaaattct-3' |
| | | | (SEQ ID NO: 16) |
| | | | P: 5'-acgaccccatgcaaaggaaaccaaca-3' |
| | | | (SEQ ID NO: 17) |
| | Survivin-3B | AB154416 | F: 5'-ccagatgacgaccccatagag-3' |
| | | | (SEQ IDNO: 18) |
| | | | R: 5'-aagaactggcccttcttgga-3' |
| | | | (SEQ ID NO: 19) |
| | | | P: 5'-cattcgtccggttgcgctttcc-3' |
| | | | (SEQ ID NO: 20) |
| | Survivin-2α | | F: 5'-gctttgttttgaactgagttgtcaa-3' |
| | | | (SEQ ID NO: 21) |
| | | | R: 5'-gcaatgagggtggaaagca-3' |
| | | | (SEQ ID NO: 22) |
| | | | P: 5'-agatttgagttgcaaagacacttag |
| | | | tatgggaggg-3' (SEQ ID NO: 23) |
| | Caspase-3 | NM_032991 | F: 5'-ctggactgtggcattgagaca-3' |
| | | | (SEQ ID NO: 24) |
| | | | R: 5'-agtcggcctccatggtattt-3' |
| | | | (SEQ ID NO: 25) |
| | | | P: 5'-tggtgttgatgatgacatggcgtgtc-3' |
| | | | (SEQ ID NO: 26) |
| | Caspase-3s | | F: 5'-agaagtctaactggaaaacccaaact-3' |
| | | | (SEQ ID NO: 27) |
| | | | R: 5'-caaagcgactggatgaacca-3' |
| | | | (SEQ ID NO: 28) |
| | | | P: 5'-attattcaggttattattatggcg-3' |
| | | | (SEQ ID NO: 29) |
| | Casp8AP2 | NM_012115 | Hs00201640_m1 |
| | Caspase-9 | NM_032996 | Hs00154261_m1 |
| | ASC | NM_013258 | Hs0154724_gH |
| | Fasl | NM_000639 | Hs00899442_m1 |
| | LTBR | NM_002342 | Hs00158922_m1 |
| | HSP90 | NM_001040141 | Hs00743767_sH |
| | TRAF5 | NM_004619 | Hs01072220_m1 |
| | BCL-x | NM_001191 | Hs00236329_m1 |
| | CD40 | NM_000074 | Hs99999100_s1 |
| House- | 18S | x03205.1 | Hs99999901_sl |
| keeping | | | |

F, forward;
NCBI, National Center for Biotechnology Information;
P, probe;
R, reverse

Survivin, caspase-3, and their splice variant expressions were determined by design primers and probes labeled at the 5′ end with FAM and at the 3′ end with TAMRA. Assays on Demand (Applied Biosystems) were used for the other studied genes. Amplifications were performed in a total volume of 25 μl in the presence of 12.5 μl Universal Master Mix (Applied Biosystems), 150 nM of each primer and 200 nM probe for survivin, 300 nM primers and 150 nM probes for survivin-DEx3, 300 nM primers and 200 nM probe for survivin-2B, 300 nM primers and 150 nM probe for survivin-3B, 600 nM primers and 200 nM probe for survivin-2a, caspase-3, and their splice variants caspase-3s, or 1.25 μl of Assays on Demand and 12 ng cDNA (or water as a negative control). The PCR program consisted of a 10-min initial denaturation step at 95° C., followed by 40 cycles of 15 sec at 95° C., and 1 min at 60° C. cDNA from MCF-7 cells was analyzed simultaneously as a control and all samples were amplified in duplicate, with results analyzed using either the 2-DCt method for expression comparison or the 2-DDCt method (Livak et al, 2001) for statistical analysis.

The Mann-Whitney U and Chi2 tests were used to compare gene expression with pathological response, with a threshold corresponding to normal mammary tissue relative expression value used to separate the population into two groups. Statistical significance was considered when p<0.05.

Microarray Experiment

Microarray analyses were performed using the Affymetrix-Microarray Platform of the Institute of Genetics and Molecular and Cellular Biology (IGBMC) and Génopole Alsace-Lorraine (Dr Philippe Kastner). The analysis used samples from 25 patients (11 with pCR and 14 with non-pCR) and the resulting profile was validated using an independent and blinded group of 13 patients (four with pCR and nine with non-pCR).

The fluorescent nucleic acids hybridized onto the microarrays were prepared from total RNA. One microgram of total RNA was reverse transcribed into cDNA using a poly-dT with an extended region as a 3′ end primer. After second-strand synthesis, all the different double-strand cDNAs had a common 3′ end extension, which was used as a specific annealing site during PCR amplification. This unidirectional PCR amplification produced single-strand linear PCR products, which were labeled by random priming with dUTP-Cy5 (red) for the test samples or with dUTP-Cy3 (green) for the reference samples. Test and reference samples were co-hybridized onto microarrays. Human microarrays from the Affymetrix-Microarray Platform of the IGBMC and Génopole Alsace-Lorraine were used, onto which 25,000 genes were spotted. Reference genes were eliminated. Hybridized slides were scanned to detect fluorescence signals at high resolution. Fluorescent intensities were normalized and standardized by using the IGBMC in-house ‘Elea’ software followed by a LOWESS (LOcal Weighted Estimates of Smooth Scatterplots) fitting-based method. Briefly, genes were selected as invariants from ranks of values in the Cy3 and Cy5 channels, and were then used in the LOWESS algorithm to compute the normalization factor between the two channel values. This generated two values: the signal value A=Log 2(test value*reference value)/2; and the log ratio M=Log 2(test value/reference value).

Microarray Data Analysis

Using the A values, the inventors determined the lowest median expression level of the population and excluded every gene with an A value lower than this. Using this heuristic filtering, we identified 14,829 genes to analyze further. From this subset of genes, statistical filtering was performed on the M values using IGBMC in-house statistical ‘Zoe’ software. The Mann-Whitney U test was then used with 1000 permutations to compare pCR and non-pCR rates, with p<0.002 considered significant.

Results

Analysis of Selected Gene Expression by Quantitative RT-PCR

When the relative expression of genes associated with cell cycle progression was compared with pathological response, it was found that the expression of these genes did not correlate with the observed pathological response, as shown by the Chi2 test. The inventors next compared the relative expression of DNA repair genes with pathological response, and the results similarly showed that the expression of these genes did not correlate with pathological response to a trastuzumab-docetaxel-based regimen. No relationship was also found between the relative expression of apoptotic genes and pathological responses. However, the Chi2 test did show a significant (p<0.0001) inverse relationship between expression of bcl-xL and response to trastuzumab-docetaxel-based treatment.

Microarray Data Analysis

Of the 25 patients in the training set, 11 (44%) patients showed pCR and 14 (56%) had non-pCR. Microarray analysis of tumor samples from these patients indicated that expression significantly differed between pCR and non-pCR tumor samples for 28 genes. Among these 28 genes, 12 were more highly expressed in pCR tumor samples (WEE1, ZNF146, SENP7, GPR22, SYNCRIP, SLC30A6, GRHL2, CCDC123, STX1A, cDNA FLJ11973 fis, clone HEMBB1001221, KIAA1549 and Homo sapiens hypothetical LOC340171), and 16 genes were highly expressed in non-pCR samples (LOC158402, PITPNA, PPP2CA, SLC35A4, NFE2L1, C5orf3, PEX19, P2RX1, CDC14A, SENP8, PSMD11, CTNS, PRKACA, LAMA3, FLJ20160, and DERL1 (see Table B). In addition, there was no difference observed for treatment effect (TAXHER01 or GETNA01) on this 28-gene expression profile.

The discriminatory 28-gene profile was then validated using the independent cohort of 13 patients. The analysis of the profile was performed without prior knowledge of the patients' pathological response. Each patient's tumor sample was classified using a correlation coefficient based on the mean expression value of each selected gene for the pCR and non-pCR subsets. A patient was classified as being in the pCR group when their correlation coefficient was higher, with mean values above the non-pCR values, and vice versa. Using this approach, the present 28-gene profile correctly classified the four pCR patients as having the pCR expression profile, and 8/9 non-pCR patients into the non-pCR profile. Thus, the present 28-gene profile for a trastuzumab-docetaxel-based regimen exhibited 100% sensibility, 89% specificity, and 92% accuracy (Table 4).

TABLE 4
Performance of the 28-gene (Table B) expression profile
for the independent cohort response prediction

| | | Predicted | | |
|---|---|---|---|---|
| | | pCR | Non-pCR | Total |
| Observed | pCR | 4 | 0 | 4 |
| | Non-pCR | 1 | 8 | 9 |
| | Total | 5 | 8 | 13 |

| | Cases | Percentage |
|---|---|---|
| Sensitivity | 4/4 | 100 |
| Specificity | 8/9 | 89 |
| Positive prediction value | 4/5 | 80 |
| Negative prediction value | 8/8 | 100 |
| Accuracy | 12/13 | 92 |

Non-pCR, non-pathological complete response;
pCR, pathological complete response

Conclusion

Using microarray analysis, the inventors generated a 28-gene profile that can discriminate between tumor samples that would attain a pCR and those that would not in response to treatment with a trastuzumab-docetaxel-based regimen, with 92% accuracy. This profile was not affected by treatment effect (TAXHER01 or GETNA01), and the results confirm previous analyses from these two studies that have commented on the association between pCR and HER2 amplification (Arnould et al, 2007).

Importantly, the present results also teach that genes not involved in classical cancer pathways such as apoptosis, cell cycle progression, or DNA repair are involved in determining responses to a trastuzumab-docetaxel-based regimen.

### Example 2

### Definitions of Subgroups of Genes

Using the same method as the one described in Example 1, the inventors showed that subgroups of genes were discriminative too.

TABLE 5
Performance of the expression profile of genes of Table C-
subgroup for the independent cohort response prediction

| | | Predicted | | |
|---|---|---|---|---|
| | | pCR | Non-pCR | Total |
| Observed | pCR | 4 | 0 | 4 |
| | Non-pCR | 2 | 7 | 9 |
| | Total | 6 | 7 | 13 |

| | Cases | Percentage |
|---|---|---|
| Sensitivity | 4/4 | 100 |
| Specificity | 7/9 | 78 |
| Positive prediction value | 4/6 | 67 |
| Negative prediction value | 7/7 | 100 |
| Accuracy | 11/13 | 85 |

TABLE 6
Performance of the expression profile of genes of Table A-
subgroup for the independent cohort response prediction

| | | Predicted | | |
|---|---|---|---|---|
| | | pCR | Non-pCR | Total |
| Observed | pCR | 4 | 0 | 4 |
| | Non-pCR | 2 | 7 | 9 |
| | Total | 6 | 7 | 13 |

| | Cases | Percentage |
|---|---|---|
| Sensitivity | 4/4 | 100 |
| Specificity | 7/9 | 78 |
| Positive prediction value | 4/6 | 67 |
| Negative prediction value | 7/7 | 100 |
| Accuracy | 11/13 | 85 |

### REFERENCES

Arnal M, Franco N, Fargeot P, Riedinger J M, Brunet-Lecomte P, Lizard-Nacol S. Enhancement of mdr1 gene expression in normal tissue adjacent to advanced breast cancer. Breast Cancer Res Treat 2000; 61:13-20.
Arnould L, Arveux P, Couturier J, Gelly-Marty M, Loustalot C, Ettore F et al. Pathologic complete response to trastuzumab-based neoadjuvant therapy is related to the level of HER-2 amplification. Clin Cancer Res 2007; 13:6404-9.
Buzdar A U, Ibrahim N K, Francis D, Booser D J, Thomas E S, Theriault R L et al. Significantly higher pathologic complete remission rate after neoadjuvant therapy with trastuzumab, paclitaxel, and epirubicin chemotherapy: results of a randomized trial in human epidermal growth factor receptor 2-positive operable breast cancer. J Clin Oncol 2005; 23:3676-85.
Chevallier B, Roche H, Olivier J P, Chollet P, Hurteloup P. Inflammatory breast cancer. Pilot study of intensive induction chemotherapy (FEC-HD) results in a high histologic response rate. Am J Clin Oncol 1993; 16:223-8.
Cobleigh M A, Vogel C L, Tripathy D, Robert N J, Scholl S, Fehrenbacher L et al. Multinational study of the efficacy and safety of humanized anti-HER2 monoclonal antibody in women who have HER2-overexpressing metastatic breast cancer that has progressed after chemotherapy for metastatic disease. J Clin Oncol 1999; 17:2639-48.
Coudert B P, Arnould L, Moreau L, Chollet P, Weber B, Vanlemmens L et al. Pre-operative systemic (neo-adjuvant) therapy with trastuzumab and docetaxel for HER2-overexpressing stage II or III breast cancer: results of a multicenter phase II trial. Ann Oncol 2006; 17:409-14.
Coudert B P, Largillier R, Arnould L, Chollet P, Campone M, Coeffic D et al. Multicenter phase II trial of neoadjuvant therapy with trastuzumab, docetaxel, and carboplatin for human epidermal growth factor receptor-2-overexpressing stage II or III breast cancer: results of the GETN(A)-1 trial. J Clin Oncol 2007; 25:2678-84.
Harris L N, You F, Schnitt S J, Witkiewicz A, Lu X, Sgroi D et al. Predictors of resistance to preoperative trastuzumab and vinorelbine for HER2-positive early breast cancer. Clin Cancer Res 2007; 13:1198-207.
Hoheisel, Nat Rev Genet. 2006 March; 7(3):200-10
Livak K J, Schmittgen T D. Analysis of relative gene expression data using real-time quantitative PCR and the 2(-Delta Delta C(T)) Method. Methods 2001; 25:402-8.

1-17. (canceled)

18. An in vitro method for predicting whether a patient would be responsive to a treatment with an anti-HER2 antibody or a HER2 blocking agent comprising determining the expression level of at least 4 genes in a biological sample of said patient, wherein said genes are GPR22, PEX19, GRHL2 and DERL1.

19. The method of claim 18, wherein the patient has a HER2-positive cancer.

20. The method of claim 18, wherein the patient has breast cancer.

21. The method of claim 18, wherein the HER2 blocking agent is an anti-HER2 antibody.

22. The method of claim 21, wherein the anti-HER2 antibody is trastuzumab.

23. The method of claim 18, wherein said method predicts whether a patient would be responsive to a treatment with a HER2 blocking agent in combination with a taxane.

24. The method of claim 18, further comprising the step of comparing the combined expression level of said genes with reference values obtained from responder and non-responder groups of patients.

25. The method of claim 18, wherein the biological sample is a diseased tissue sample.

26. The method of claim 18, wherein the expression level is determined by quantifying the level of mRNA of said genes in the biological sample.

27. The method of claim 26, wherein the expression level is determined by real-time quantitative or semi-quantitative RT-PCR.

28. The method of claim 26, wherein the expression level is determined using a DNA chip.

29. The method of claim 18, further comprising determining the expression level of the transcripts listed in Table B, or of a subcombination thereof.

30. The method of claim 26, further comprising determining the expression level of any or all of the transcripts listed in Table C.

31. A DNA chip comprising a solid support which comprises nucleic acids that are specific to GPR22, PEX19, GRHL2 and DERL1 genes.

32. The chip of claim 31, said chip further comprising nucleic acids that are specific to any or all of the transcripts listed in Table B, or a subcombination thereof.

33. The chip of claim 32, said chip further comprising nucleic acids that are specific to any or all of the transcripts listed in Table C.

34. A method for treating a patient with a HER2-related disease,
which method comprises:
predicting whether a patient would be responsive to a treatment with an anti-HER2 antibody, which method comprises determining the expression level of at least 4 genes in a biological sample of said patient, wherein said genes are GPR22, PEX19, GRHL2 and DERL1, which classifies as responder to a treatment with a HER2 blocking agent and administering a HER2 specific antibody to a patient classified as a responder.

35. The method of claim 34, wherein the HER2-related disease is cancer.
